(19) United States
(12) Patent Application Publication
(10) Pub. No.: US 2018/0169292 A1
(43) Pub. Date: Jun. 21, 2018

(54) SILICONE WOUND DRESSING AND METHODS FOR MANUFACTURING AND USING THEREOF

(71) Applicant: Bio Interface Engineering, L.L.C., Columbia, MO (US)

(72) Inventors: Hirotsugu Yasuda, Columbia, MO (US); Werner Akira Yasuda, Columbia, MO (US)

(21) Appl. No.: 15/381,192

(22) Filed: Dec. 16, 2016

(51) Int. Cl. A61L 15/26; A61L 15/18; A61F 13/00

(52) U.S. Cl. CPC A61L 15/26; A61L 15/18; A61F 13/00017; A61F 2013/00217; A61F 13/00987; A61F 13/00046; A61L 2300/108; A61F 13/00063

(57) ABSTRACT

Provided are a silicone wound dressing, a method of manufacturing thereof and a method of using thereof. The silicone wound dressing has a silicone dressing base material and an amorphous carbon coating formed on the silicone dressing base material. The amorphous carbon coating is formed by subjecting the silicone dressing base material to a plasma polymerization in a plasma device under an atmosphere of a mixed gas comprising a hydrocarbon gas and an oxygen-containing gas. The silicone wound dressing has excellent oxygen permeability and is less likely to adhere to the wound. The speedy recovery of the wound is promoted by covering the wound with the silicone wound dressing.

### BACKGROUND OF THE INVENTION

### Field of the Invention

The present invention relates generally to a silicone wound dressing and a method for manufacturing and its usage thereof. In particular, the wound dressing comprises of silicone base materials and an amorphous carbon coating over the silicone base materials. The amorphous carbon coating of the silicone wound dressing is formed by a plasma polymerization of a gas mixture containing hydrocarbon gas and, oxygen containing gas. The silicone wound dressing has properties of superior oxygen permeability and non-adhesion to wounds. A speedy recovery of wounds is promoted by covering the wounds by the coated silicone wound dressing.

### Description of the Related Art

Many types of wound dressings are commercially available. Dressings using gauze to absorb the fluid from wounds to make the wounds dry are a typical type of dressing. However, gauze dressing has a strong tendency to stick to the wounds due to drying and to damage the reconstructed tissue when the dressings are changed. In addition, since it does not seal the wounds tightly, such dressings have a risk of infection from outside bacteria.

On the other hand, dressings using urethane and silicone resin are also commercially available. The urethane dressing can cover the wounds tightly to prevent infection from the pathogen outside; however the urethane cannot have absorbing capability of the fluid from wounds. And, since the urethane has poor oxygen permeability, it is stressful to the wounds, which may slow down the healing process of wound.

Silicone dressings are highly oxygen permeable and resilient, which can provide condition necessary for rapid healing of wounds. However, silicone is well known for absorption of wound body fluids, which may attract bacteria for infection. Furthermore, the surface of silicone is very sticky, which can promote the silicone's adhesion to wounds.

There have been advanced dressings developed to promote an early wound recovery by combining these dressing materials with absorbents such as hydrogels and hydrocolloids. The absorbents are used to take the wound fluids for smooth recovery of the wounds. In addition, the absorbents are also mixed with antibiotics to prevent risk of infection by bacteria. In order to prevent dressing adhesion to wound, additional supporting materials such as polymeric mesh contacting the absorbents are used. This is to minimize the surface contact area of the dressing to the wounds. However, no surface treatment directly applied to the dressing material itself in order to avoid dressing adhesion has been developed. Therefore, there has still been a strong demand to develop a biocompatible dressing capable of avoiding the risk of bacterial infection and non-adhesion to wounds in order to facilitate patient comfort and speedy tissue reconstruction.

On the other hand, it has been reported that surface treatment such as amorphous carbon coating on a highly oxygen permeable material is effective in promoting both areal reduction of bacteria growth and biocompatibility. Such a coating is also known to reduce the surface tackiness and the absorption of proteins and lipids. However, such a coating has not been mentioned to the area of wound dressing development.
Prior Art Document 1: Journal of Athletic Training: 30, 143-146(1995)

For an early recovery of wounds, it is necessary to cover wounds tight to avoid a risk of infection from pathogen outside. It also mentioned that it is important to maintain wounds from desiccation.
Prior Art Document 2: Contact Lens Spectrum, January (2002)

An extended wear contact lens using silicone has shown that less bacteria growth when it is treated with amorphous carbon coating. It also indicated that amorphous carbon coating is biocompatible.
Prior Art Document 3: Luminous Chemical Vapor Deposition & Interface Engineering: CRC Press, 2004 chapter 35, p 1-23

The biocompatible nature of an amorphous carbon coating is fully discussed with an extended wear silicone contact lens treated by amorphous carbon coating. The reduction of silicon surface stickiness reduction by amorphous carbon coating is fully described.
Prior Art Document 4: Progress in Organic Coatings; 74, 667-678(2012)

Adhesion of E coli bacteria was reduced at a surface treated by amorphous carbon coating.
Prior Art Document 5: U.S. Pat. No. 5,681,579

The wound fluid is absorbed by a dressing containing a hydrocolloid supported by a hydrophobic polymer. The flexible dressing material with an adhesive larger than that of absorbent seals the wound tight to prevent from contamination and bacteria outside.
Prior Art Document 6; U.S. Pat. No. 5,336,209A

Hydrocolloids are used as absorbent, which is supported by polymeric mesh. The polymeric mesh controls the wound fluid transport as well as water vapor. The contact area of the polymeric mesh has a function to non-adherent.

### BRIEF SUMMARY OF THE INVENTION

This invention is related to the manufacturing method of a silicone wound dressing and its usage of the modified silicone wound dressing. Specifically it is related to the manufacturing method and the usage of a silicone wound dressing having properties of superior oxygen permeability and non-adhesion to wounds.

The objective of this invention is to minimize the interaction between the wounds and a wound dressing surface by making the wound dressing non-adhesive to a wound. The normal surface tackiness of the silicone wound dressing material is reduced by covering the silicone wound dressing material base with an amorphous carbon coating, which has a tight network structure. This structure prevents the wound dressing adhering to wounds, thereby also preventing damage to the reconstructed tissue when the dressing is removed.

The amorphous carbon coating can control the uptake of body fluid from wounds and maintain an appropriate moisture level of wounds by controlling the water evaporation from the wounds. This can establish a comfortable wound recovery condition by preventing an excess drying of the wounds.

The purpose of this invention is to provide a wound dressing, which by means of an amorphous carbon coating prevents the dressing adherence to wounds and resists bacterial infection while maintaining a natural skin metabolism made possible by the high oxygen permeability of the dressing's silicone base material.

Another purpose of this invention is to provide a manufacturing method for the silicone wound dressing.

Another purpose of this invention is to provide a speedy wound recovery method by covering the wounds with the silicone wound dressing.

In one aspect of this invention, a silicone wound dressing comprises of a silicone dressing base material and an amorphous carbon coating. In one embodiment, the amorphous carbon coating is formed by exposing the silicone dressing base material to the plasma polymerization of a gas mixture of a hydrocarbon gas and an oxygen containing gas. In another embodiment, the hydrocarbon gas is methane. In another embodiment, the gas mixture includes rare gases. In another embodiment, the silicone dressing base material additionally includes a hardened type of silicone rubber. The silicone dressing base material includes a peroxide-hardened type silicone rubber. In another embodiment, the thickness of the amorphous carbon coating is between 5 nm and 100 nm. In another embodiment, the oxygen permeability of the silicone wound dressing is 100 nm barrier or more at the wound dressing base material thickness spans a range of 50 □m to 2000 □m.

In one aspect of this invention, a silicone wound dressing manufacturing method is described. The silicone wound dressing comprises of a silicone dressing base material and an amorphous carbon coating. The method includes a process of the amorphous carbon coating formation by exposing the silicone base material to a plasma polymerization of a gas mixture of a hydrocarbon gas and oxygen-containing gas. In one embodiment, the hydrocarbon gas is methane. In another embodiment, the oxygen containing gas is dry air or oxygen. In another embodiment, the gas mixture includes rare gases. In another embodiment, the hydrocarbon gas is methane and the oxygen containing gas is a dry air. In another embodiment, the volume mixture ratio of methane and dry air is between 50:50 and 80:20. In another embodiment, the ultimate vacuum level of plasma the polymerization device is 0.2 Pa or less.

In one aspect of the invention, the usage of silicone wound dressing, which promotes a speedy wound recovery, is provided. The silicone wound dressing comprises of a silicone dressing material base and an amorphous carbon coating formed on the silicone dressing material base. This method includes a process to cover the wounds by the silicone wound dressing.

In this invention, a silicone wound dressing with a significantly improved comfort usage is provided by high oxygen permeability and resilience.

The surface of the silicone wound dressing with an amorphous carbon coating is non-adhesive to wounds and can resist against bacterial growth, which can promote a speedy recovery of wounds.

### DETAILED DESCRIPTION OF THE INVENTION

The novel silicone wound dressing of this invention is formed by subjecting a silicone dressing base material, which is obtained by curing an addition hardened-type silicone rubber or a peroxide hardened silicone rubber, to a plasma polymerization under an atmosphere of a gas mixture comprising of a hydrocarbon gas and an oxygen containing gas to form an amorphous carbon coating on its surface.

The following illustrates the details of the present invention:

Manufacturing Method of Silicone Dressing Base

Components (A)

The component (A) is an organopolysiloxane having at least two alkenyl groups preferably 2-5 groups bonded to the silicon atom. Its location may be anywhere in the organopolysiloxane. The degree of polymerization is between 10 and 10000 preferably between 100 and 8000. When the degree of polymerization exceeds more than 10000, the manufacturing ability is decreased.

The representative examples of the alkenyl groups bond to the silicon atom are vinyl group, allyl group, butenyl group, pentenyl group, hexenyl group, heptenyl group, preferably vinyl group.

In the component (A) organopolysiloxnane, the organic groups bond to the silicon atom other than the alkenyl group are substituted or unsubstituted hydrocarbons having no unsaturated aliphatic group. The representative examples of the unsubstituted hydrocarbon groups are methyl group, ethyl group, n-propyle group, octyl group, cyclohexyl group, and phenyl group. The representative examples of the substituted hydrocarbon groups are tolyl group, xylyl group, benzyl group. A methyl group other than the alkenyl group is preferred.

The component (A) can be used as a single component, or two or more variants can be mixed.

Component (B)

Component (B) is shown as an average unit formula (1) having at least 2 hydrogen atoms (SiH) per unit molecule, preferably organohydrogenpolysiloxane with 3 or more atoms.

R¹ₐH_{b}SiO_{(4-a-b)/2} (1)

In the formula, R¹ is an unsubstituted or substituted univalent hydrocarbon group and a should be 0.7≤a≤2.1 and b should be 0.001≤b≤1.0, and a+b should span the range of 0.8≤a+b≤3.0.

In the above formula (1), R¹ groups are substituted or unsubstituted hydrocarbons having no unsaturated aliphatic group. The representative examples of the unsubstituted hydrocarbon groups are methyl group, ethyl group, n-propyle group, octyl group, cyclohexyl group, and phenyl group. The representative examples of the substituted hydrocarbon groups are tolyl group, xyly group, benzyl group. A methyl group other than the alkenyl group is preferred.

The hydrogen atom bond to the silicon atom (SiH), which is at least 2 per molecule and preferably 3 or more can be located at the end or within the unit or can be located in both positions. The structure of organohydrogenpolysiloxane is linear, cyclic, branched, or three dimensional and the degree of polymerization per one molecule is normally between 2-400 and preferably between 4 and 100.

The component (B) can be used alone or two or more variants can be mixed.

The mixing amount of component (B) is such an amount that the number of the hydrogen atoms bonded to the silicon atom in the component (B) is in the range of 0.5 to 5.0, preferably 1 to 5 for one alkenyl group bonded to the silicon atom in the component (i).

If the blended amount of the component (B) is such an amount that the number of the hydrogen atoms bonded to the silicon atom in component (B) is less than 0.8 for one alkenyl group bonded to the silicon atom in the component (A), the resulting composition will not be sufficiently well cured. Also, If the blended amount of component (B) is such an amount that the number of the hydrogen atoms bonded to the silicon atom in component (B) is more than 10 for one alkenyl group bonded to the silicon atom in the component (A), the resulting silicone rubber will have extremely poor rubber elasticity.

Component (C)

The component (C), which is an additional reaction catalyst, may be any catalyst which accelerates the addition reaction of the alkenyl group in the component (A) with the hydrogen atom bonded to the silicon atom in the component (B). The specific examples of these include platinum group metals and their compounds including platinum, palladium, rhodium, and the like; an alcohol-modified chloroplatinic acid; a coordination compound of chloroplatinic acid with an olefin, vinyl siloxane or an acetylene compound; tetrakis(triphenylphosphine)palladium; and chlorotris(triphenylphosphine) rhodium; and the like, with platinum group compounds being especially preferred.

The component (C) may be used alone, or two or more variants of components (C) may be used in combination.

The blended amount of the component (C) may be any effective amount as the catalyst, and preferably be in the range of 0.1 to 1000 ppm, more preferably 3 to 100 ppm based on the mass converted into the catalyst metal elements for the total amount of the Components (A) and (B). If the amount of component (C) is within the range, the reaction rate of the addition reaction will be appropriate and the cured material will have a good heat resistance.

When curing the silicone wound dressing base material by an addition reaction, additives such as methylvinylcyclotetrasiloxane, an acetylene alcohol or an ethnylcyclohenanol may be added in order to obtain a good storage stability at room temperature and suitable a pot life.

In addition, curing the silicone wound dressing base by the addition reaction may be carried out by heating the base at a temperature of 60 to 250° C. for about one minute to five hours.

Peroxide Curing Agents

The peroxides of the component (D) include benzoyl peroxide, t-butyl perbenzoate, o-methyl benzoyl peroxide, p-methyl benzoyl peroxide, di-t-butyl peroxide, dicumyl peroxide, 1,1-bis(t-butyl peroxy)-3,3,5-trimethyl cyclohexane, 2,5-dimethyl-2,5-di(butyl peroxy)hexane, 2,5-dimethyl-2,5-di(butyl peroxy)hexyne, 1,6-bis(p-tolyl peroxy carbonyloxy)hexane, di(4-methyl benzoyl peroxy)hexamethylene biscarbonate, and the like. Any of these components may be used alone, or two or more of these may be used in combination. The additional amount of component (D) may be 0.01 to 5 parts by mass, preferably 0.05 to 3 parts by mass for 100 parts by mass of component (A).

The curing condition for the composition using the peroxide is not specifically limited, but the composition may be cured by heating it at a temperature of 100 to 300° C. for about one minute to five hours.

Other components that may be blended include finely powder silica as a mechanical reinforcement, heat resistance improver, pigment etc.

The silicone wound dressing base is prepared by any kind of conventional techniques (for example, the cast molding manufacturing method, the calendar roll manufacturing method, the injection mold manufacturing method, press manufacturing method etc. . . . ). The thickness of the silicone wound dressing base is between 50 μm-2000 μm preferably between 100-1000 μm. In case that the thickness is below 50 μm, it may not be sufficient enough as the dressing material and if it is more than 2000 μm, the dressing handling may become difficult and it may not cover wounds tightly. The silicone dressing base material is exposed to a plasma polymerization to obtain a silicone wound dressing having an amorphous carbon coating.

Plasma Polymerization Treatment of Silicone Dressing Base Material

According to the present invention, an amorphous carbon coating is formed on the surface of the above-described dressing base material. The amorphous carbon coating is formed by a plasma polymerization treatment under an atmosphere of a mixed gas of a hydrocarbon gas and an oxygen-containing gas, preferably a mixed gas of methane and dry air. Specifically, the plasma polymerization treatment under an atmosphere of a mixed gas of methane and dry air is conducted by placing the silicone dressing base material into a plasma polymerization device and thereafter purging the device to an attainable pressure less than a predetermined threshold. In the case of placing the silicone dressing base material into the vacuum device and purging the device, the gas and moisture adsorbed in the silicone dressing base material to be treated is discharged together with the adsorbed gas on the surface of the device, the occluded gas inside the device and the discharged gas from the sealing material. Accordingly, it is practically and commercially preferable to make the attainable pressure of the device constant in order to reduce quality fluctuations between and within treated lots. The attainable pressure during the vacuum purging process is preferably 0.5 Pa or less, more preferably 0.2 Pa or less. If it is 0.5 Pa or less, the fluctuation of the coated film thickness between and within treated lots due to the influence of the adsorbed gas on the surface of the device, the gas adsorbed into the silicone dressing base material, etc., will be preferably reduced, as discussed above. In order to purge the device to the predetermined range, a vacuum pump which is capable of purging the device to the targeted degree of vacuum may be used. Any types of generally known pumps such as a sealed rotary pump and a dry pump may be used. Also, a measuring instrument for measuring the degree of vacuum inside the device may be any type of vacuum gauge which can measure the predetermined range of the pressure, including for example a diaphragm vacuum gauge, a Pirani vacuum gauge, and the like. Moreover, according to the present invention, it is preferable to load the silicone dressing base material onto a holding jig and the holding jigs further placed on a supporting rotation wheel which rotates through the plasma zone repeatedly during the amorphous carbon coating process in order to treat the surface of the silicone dressing base material uniformly and efficiently. The material of the jig can be any materials which are generally used in a vacuum device, for example, stainless steel.

The mixture ratio of methane to dry air (methane:dry air) that can be used in the plasma polymerization treatment is preferably 50:50 to 80:20 by volume. If the amount of dry air is greater than this ratio, the formation speed of the film which is formed on the silicone dressing base material will be undesirably decreased (resulting in an increase of needed treatment time). If the amount of methane is greater than this ratio, the film which is formed on the surface of the silicone dressing base material becomes brittle. The above-described gas mixture ratio is more preferably 66:34 to 75:25.

A mixed gas of methane and dry air may be introduced into the device, or a pre-mixture of methane and dry air (the water content of 3 ppm) may introduced into the device for amorphous carbon coating. In the first step, it is preferable to continuously supply the gas into the device to conduct the plasma polymerization treatment while maintaining the pressure inside the device constant by the vacuum pump. The flow rate of the mixed gas of methane and dry air introduced into the device is preferably 1.5 to 20 sccm for the chamber volume of 100-700 L, more preferably 2 to 10 sccm for the chamber volume of 150 to 600 L.

It is preferable to conduct the plasma polymerization treatment after the gas is introduced into the device and the pressure inside the device is stabilized. The treatment conditions during the electrical discharge should be suitably selected, and for example, it is preferable that the pressure inside the device is between 3 to 10 Pa, the discharge input is 10 to 80 W and the electrical power source for the plasma generation has a low frequency of about 6 to 15 kHz. Also, the device may be of an inner electrode type, an outer electrode type, and the like, but any known device can be used for carrying out the treatment. The plasma polymerization treatment time in the first step may be set in consideration of the desired thickness, and may be, for example, 3 to 30 minutes, preferably 5 to 20 minutes.

According to the above steps, an amorphous carbon coating can be formed on the surface of the silicone dressing base material. It is preferable that the amorphous carbon coating is formed on the entire surface of the base material. The thickness of the amorphous carbon coating is critical in order to make a good surface barrier at the surface of the silicone dressing base material. The thickness of the film can be measured by using an automatic ellipsometer. Instead of direct measurement of the amorphous carbon coating at the surface of the silicone dressing base material, the amorphous carbon coating thickness is measured at the same deposit at a silicon wafer by the ellipsometer. A few silicon wafers are mounted in any area on the holding jig and the thickness of the amorphous carbon coating formed on a silicon wafer is measured, whereby the measured film thickness can be regarded as the equivalent thickness of the coated film formed on the silicone dressing base material. The thickness of the coated film is preferably 5 nm to 100 nm, more preferably 10 nm to 50 nm. If the thickness of the coated film is 5 nm or more, the amorphous carbon coating will have sufficient strength, and, if the thickness of the coated film is 100 nm or less, the resulting silicone wound dressing will have high oxygen permeability.

Method for Manufacturing Silicone Wound Dressing

The silicone-based wound dressing of the present invention can be autoclaved for sterilization. Any undesirable elements such as impurities or organisms which may be present in the silicone wound dressing-based material can also be extracted with organic solvents to remove them.

Usage of Silicone Wound Dressing

In this invention, the silicone wound dressing can promote a speedy recovery of wounds by covering the wounds. When covering the wounds by silicone wound dressing, it can be used to adjust the dressing to the desired size of wounds or it can be wrapped around the wounds. It can promote a speedy recovery of wounds by sealing the wounds completely and preventing bacterial intrusion and growth from the outside.

The following illustrate practical examples of the present invention, but not to represent limits of practical examples.

<Preparation of Composition a Using Addition Type Cured Silicone Rubber>

A linear organopolysiloxane containing vinyl group as the component (A) is shown in (2)

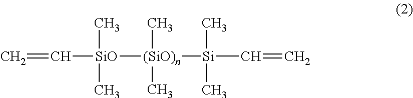

(n=350 as an average polymerization degree)

As the component (B), organohydrogenpolysiloxane is shown in (3)

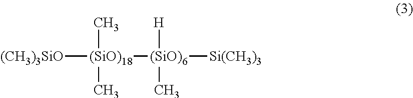

As the component (C), a complex of platinum-divinylytetramethyldisiloxane (platinum content 0.5 weight %)
As other component, ethynylcyclohexanol as curing control agent.

Component (A) 100 parts of organopolysiloxane as shown in (2), component (1) 5 parts of organohydrogenpolysiloxane as shown in (3), component (C), 0.2 part of curing control agent, ethylcylohexanol are mixed and degassed and then poured into a mould (100 m thickness) and heated at 150 deg C. for 1 hour. A silicone wound dressing base material A having the thickness of 100 mm and hardness of 60 (Type A 60) was prepared

<Preparation of Composition B Using Peroxide-Hardened Silicone Rubber>

As composition (A), linear organopolysiloxane is shown in (4).

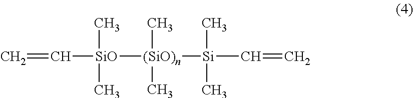

(n=8000 as an average polymerization degree)

As the component (D), 2,5-dimethyl-2, 5-bis(t-butyl peroxide) hexane
As other component, fine silica powder (surface area of 150 m²/g by BET Method)
Component (A) 100 parts of organopolysiloxane as shown in (4), component (D) 1 part of 2,5-dimethyl-2, 5-bis(t-butyl peroxide) hexane, 50 parts of fine silica powder are mixed and a sheet at the thickness of 100 □m was prepared by calendar roll method. After a post curing at 200 deg C. for 4 hours, a silicone dressing base material at the thickness of 100 m and hardness of 50 (Type A 50) was obtained as silicone wound dressing base material B.

<Preparation of Silicone Wound Dressing with Methane Plasma Treatment>

A “plasma polymerization device” manufactured by Shinko Seiki Co. Ltd. was used for the methane plasma treatment. The silicone dressing base materials. A and B to be subjected to the plasma treatment was arranged on the holding jig and placed inside a bell jar (a reaction vessel: 105 L) which was evacuated to about 0.2 Pa and kept for about 10 minutes. Then, a reactive gas (a mixed gas of methane and dry air:methane, 2 to dry air, 1 by volume) was continuously introduced while maintaining the pressure of the gas mixture at a predetermined level. After that, the plasma treatment was carried out for 10 minutes at the electrical current of 0.5 A using a 15 KHz power supply to treat silicone wound dressing base materials A and B. Then, silicone wound dressings, A-1 and B-1 were obtained after autoclave.

Measurement Methods used in the examples of the present invention

Thickness of Amorphous Carbon Coating

Silicon wafers are placed on the jig during the plasma treatment process. Then, the amorphous carbon coating thickness on the silicon wafers was measured and correlated with the amorphous carbon coating thickness on the silicone dressing base material. The thickness measurements were performed by F20-UV (Filmetrics, Inc).

Oxygen Permeability Coefficient

The oxygen permeability coefficient of a silicone dressing base material was measured in water at 35° C. by using an IP1 type film oxygen permeability meter manufactured by Rika Seiki Industries Co. Ltd.

Water Contact Angle and Scratch Test

A contact angle of a pure water was measured at a temperature of 23° C. and a relative humidity (RH) of 55% with a contact angle meter CA-V manufactured by Kyowa Interface Science Co. Ltd. Surface scratch test was performed using plastic tweezers. The test surface was scratched by this tweezers five (5) times, and then the water contact angles were measured by the same method.

XPS

XPS (X ray Photon Spectroscopy) (ULVAC-PHI, Inc) was used for Si₂ₚ and C₁ₛ measurements.

Dye Test

A drop of oil red (propylene glycohol) was placed over the test sample and left for 5 minutes. Then, the drop of dye was rinsed off and the degree of staining was evaluated by eye.

Animal Test

The animal test was cried out using Yucatan female pig. The full thickness square skin wounds (2 cm×2 cm, 3 wounds per side) were created on the dorsal-lateral area of the animal. The silicone dressings sterilized by autoclave. B-1 and B were placed on the wounds and examined for 10 days.

The dressings were removed at Days 4, 7, and 10 respectively for the evaluation, and the thicknesses of the reconstructed tissue were measured at each evaluation day.

### Example 1

The results of the amorphous carbon coating thickness, oxygen permeability, water contact angle, scratch test, XPS, and dye test for the silicone wound dressing A-1 are summarized in Table 1.

### Example 2

The results of the amorphous carbon coating thickness, oxygen permeability, water contact angle, scratch test, XPS, and dye test for the silicone wound dressing B-1 are summarized in Table 1.

### Example 3

The animal test results of silicone wound dressing B-1 are summarized in Table 2.

### Comparative Example 1

The results of the oxygen permeability, water contact angle, scratch test, XPS, and dye test for the silicone wound dressing base material A are summarized in Table 1.

### Comparative Example 2

The results of the oxygen permeability, water contact angle, scratch test, XPS, and dye test for the silicone wound dressing base material 11 are summarized in Table 1.

### Comparative Example 3

The animal test results of silicone wound dressing base material B are summarized in Table 2.

TABLE 1

| | | | Comparative | Comparative |
|---|---|---|---|---|
| | | | example 1 | example 2 |
| | | Example 2 | Silicone | Silicone |
| | Example 1 | Silicone | wound | wound |
| | Silicone | wound | dressing | dressing |
| | wound | dressing | base | base |
| Attributes | dressing A-1 | B-1 | material A | material B |
| Amorphous | 20 | 20 | 0 | 0 |
| carbon coating | | | | |
| thickness (nm) | | | | |
| Oxygen | 340 | 370 | 530 | 540 |
| permeability | | | | |
| (barrer)* | | | | |
| Water contact | 55 | 57 | 119 | 121 |
| angle (°) | | | | |
| Water contact | 64 | 61 | 111 | 115 |
| angle after | | | | |
| scratch test (°) | | | | |
| XPS Si₂ₚ/C₁ₛ | 0.10 | 0.20 | 0.40 | 0.42 |
| Dye test by | No stain | No Stain | Stained | Stained |
| oil red | | | | |

*1 barrer = 1 × 10⁻¹⁰ cm³ (STP) cm/(s cm² cmHg)

TABLE 2

| | | | Comparative |
|---|---|---|---|
| | | | example 3 |
| | | Example 3 | Silicone wound |
| Evaluation | | Silicone wound | dressing, base |
| days | Evaluation Attributes | dressing B-1 | material B |
| 4 days | Adhesion to wound | None | Yes |
| | Thickness of reconstructed | 0 | 0 |
| | tissue (mm) | | |
| 7 days | Adhesion to wound | None | Yes |
| | Thickness of reconstructed | 2.0 | 0.7 |
| | tissue (mm) | | |
| 10 days | Adhesion to wound | None | None |
| | Thickness of reconstructed | 3.5 | 2.5 |
| | tissue (mm) | | |

The invention has been described with reference to the example embodiments described above. Modifications and alternations will occur to other upon a reading and understanding of this specification. Example embodiments incorporating one or more aspects of the invention are intended to include all such modifications and alternations insofar as they come within the scope of the appended claims.

1-16. (canceled)

17: A method of promoting a speedy recovery of wound, the method comprising providing a silicone wound dressing comprising a silicone dressing base material and an amorphous carbon coating formed on the silicone dressing base material, and covering the wound with the silicone wound dressing,
wherein the silicone dressing base material comprises at least one of a vinyl group-containing organopolysiloxane and an organohydrogenpolysiloxane.

18: The method according to claim 17, wherein providing the silicone wound dressing comprises forming the amorphous carbon coating by subjecting the silicone dressing base material to a plasma polymerization in a plasma device under an atmosphere of a mixed gas comprising a hydrocarbon gas and an oxygen-containing gas.

19: The method according to claim 17, wherein the silicone dressing base material comprises both a vinyl group-containing organopolysiloxane and an organohydrogenpolysiloxane.

20: The method according to claim 17, wherein the vinyl group-containing organopolysiloxane is represented by the following formula:

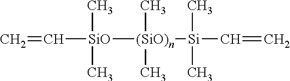

21: The method according to claim 17, wherein the organohydrogenpolysiloxane is represented by the following formula:
R¹ₐH_{b}SiO_{(4-a-b)/2}
wherein, in the formula, R¹ is an unsubstituted or substituted univalent hydrocarbon group, a is 0.7≤a≤2.1, b is 0.001≤b≤1.0, and a+b is in a range of 0.8≤a+b≤3.0.

22: The method according to claim 17, wherein the organohydrogenpolysiloxane is represented by the following formula:

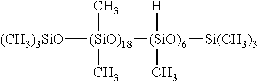

23: The method according to claim 17, wherein the silicone dressing base material further comprises a complex of platinum-divinyltetramethyldisiloxane.

24: The method according to claim 19, wherein the silicone dressing base material further comprises a complex of platinum-divinyltetramethyldisiloxane.

25: The method according to claim 17, wherein silicone dressing base material further comprises one or more peroxides.

26: The method according to claim 20, wherein silicone dressing base material further comprises one or more peroxides.
